(19) United States
(12) Patent Application Publication
(10) Pub. No.: US 2015/0351660 A1
(43) Pub. Date: Dec. 10, 2015

(54) ABSOLUTE THORACIC IMPEDANCE FOR HEART FAILURE RISK STRATIFICATION

(71) Applicant: Cardiac Pacemakers, Inc., St.Paul, MN (US)

(72) Inventors: Qi An, Blaine, MN (US); Pramodsingh Hirasingh Thakur, Woodbury, MN (US); Ramesh Wariar, Blaine, MN (US)

(21) Appl. No.: 14/711,020

(22) Filed: May 13, 2015

Related U.S. Application Data
(60) Provisional application No. 62/007,954, filed on Jun. 5, 2014.

(51) Int. Cl. A61B 5/053; A61B 5/00; A61B 5/08; A61B 5/0205; A61B 7/04

(52) U.S. Cl. CPC A61B 5/0535; A61B 5/0205; A61B 7/04; A61B 5/0809; A61B 5/7275; A61B 5/6823

(57) ABSTRACT

An apparatus may include a sensing circuit configured to generate a sensed physiological signal representative of thoracic impedance of a subject and a controller circuit. The a controller circuit is electrically coupled to the sensing circuit and includes a measurement circuit that determines a measure of absolute thoracic impedance using the sensed physiological signal, and a risk circuit that quantifies a risk of worsening heart failure (WHF) for the subject using a comparison of the determined measure of absolute thoracic impedance to a specified threshold value of absolute thoracic impedance, and generate an indication of risk of WHF of the subject according to the quantifying of the risk.

### BACKGROUND

Ambulatory medical devices include implantable medical devices (IMDs) and wearable medical devices. Some examples of these implantable medical devices (IMDs) include cardiac function management (CFM) devices such as implantable pacemakers, implantable cardioverter defibrillators (ICDs), cardiac resynchronization therapy devices (CRTs), and devices that include a combination of such capabilities. The devices can be used to treat patients or subjects using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Other examples of IMDs include implantable diagnostic devices, implantable drug delivery systems, or implantable devices with neural stimulation capability.

Wearable medical devices include wearable cardioverter defibrillators (WCDs) and wearable diagnostic devices (e.g., an ambulatory monitoring vest). WCDs can be monitoring devices that include surface electrodes. The surface electrodes are arranged to provide one or both of monitoring to provide surface electrocardiograms (ECGs) and delivering cardioverter and defibrillator shock therapy. Medical devices (e.g., implantable and wearable) can also include one or more sensors to monitor one or more physiologic parameters of a subject.

Some medical devices include one or more sensors to monitor different physiologic aspects of the patient. The devices may derive measurements of hemodynamic parameters related to chamber filling and contractions from electrical signals provided by such sensors. Sometimes patients who are prescribed these devices have experienced repeated heart failure (HF) decompensation or other events associated with worsening HF. Symptoms associated with worsening HF include pulmonary and/or peripheral edema, dilated cardionvapathy, or ventricular dilation. Some patients with chronic HF may experience an acute HF event. Device-based monitoring can identify those HF patients having a risk of experiencing an acute HF event.

### OVERVIEW

This document discusses systems, devices and methods for improved monitoring of respiratory function in patients or subjects with pulmonary conditions. An apparatus example can include a sensing circuit configured to generate a sensed physiological signal representative of thoracic impedance of a subject and a controller circuit. The controller circuit is electrically coupled to the sensing circuit and includes a measurement circuit configured to determine a measure of absolute thoracic impedance using the sensed physiological signal, and a risk circuit configured to quantify a risk of worsening heart failure (WHF) for the subject using a comparison of the determined measure of absolute thoracic impedance to a specified threshold value of absolute thoracic impedance, and generate an indication of risk of WHF of the subject according to the quantifying of the risk.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustration of portions of an example of a system that includes an implantable medical device.

FIG. 2 is an illustration of portions of another example of a system that uses an IMD.

FIG. 3 is a flow diagram of a method of operating an ambulatory medical device to assess the risk that a subject will experience a heart failure event.

FIG. 4 shows a block diagram of portions of an example of an ambulatory medical device to assess the risk that a subject will experience a heart failure event.

FIG. 5 shows a block diagram of portions of an example of an ambulatory medical device system to assess the risk that a subject will experience a heart failure event.

### DETAILED DESCRIPTION

An ambulatory medical device may include one or more of the features, structures, methods, or combinations thereof described herein. For example, an ambulatory respiration monitor may be implemented to include one or more of the advantageous features or processes described below. It is intended that such a monitor, or other implantable, partially implantable, wearable, or other ambulatory device need not include all of the features described herein, but may be implemented to include selected features that provide for unique structures or functionality. Such a device may be implemented to provide a variety of diagnostic functions.

Systems and methods are described herein for improved assessment of HF of a patient. A fraction of patients with chronic HF may experience an acute HF event (e.g., a HF decompensation event) over a time frame such as a one year period. This fraction may be small (e.g., 10% of chronic patients) with those at highest risk being even smaller (e.g., 1% of chronic patients). If health care resources are limited, it is desirable to identify those patients who are most at risk and allocate medical care resources accordingly. A device-quantified risk assessment for HF may help physicians identify those patients with an extremely high risk of HF (e.g., the 1% with the highest risk), and allocate resources for monitoring and treating HF accordingly while maintaining similar quality of health care to all HF patients.

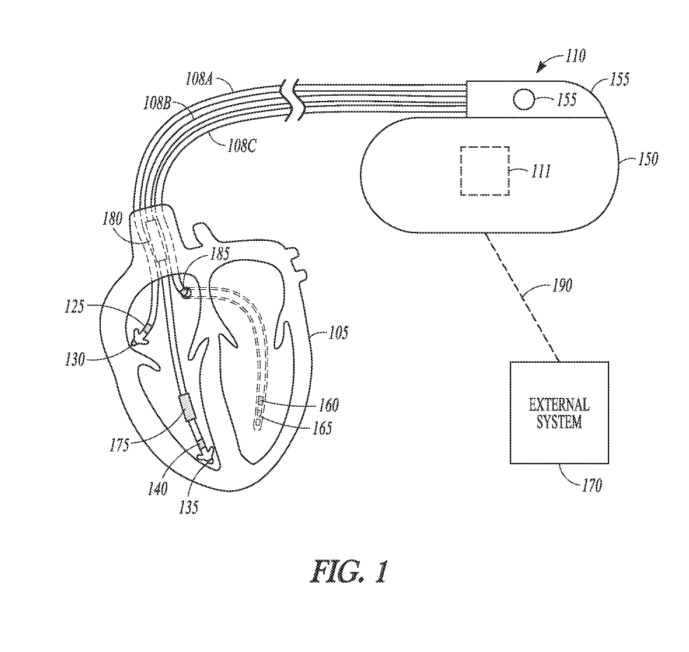

Medical electronic systems can be used to obtain information related to a patient's or subject's physiologic condition. FIG. 1 is an illustration of portions of an example of a system that includes an IMD 110. Examples of IMD 110 include, without limitation, a pacemaker, a defibrillator, a cardiac resynchronization therapy (CRT) device, an implantable diagnostic device, an implantable loop recorder, or a combination of such devices. The IMD 100 may be a neurostimulation device such as among other things, a vagus nerve stimulator, baroreflex stimulator, or a carotid sinus stimulator. The IMD 110 can be configured by shape and size for transvenous implantation or configured for subcutaneous implantation. The IMD 110 can be coupled by one or more leads 108A-C to heart 105. Cardiac leads 108A-C include a proximal end that is coupled to IMD 110 and a distal end, coupled by electrical contacts or “electrodes” to one or more portions of a heart 105. The electrodes can deliver cardioversion, defibrillation, pacing, or resynchronization therapy, or combinations thereof to at least one chamber of the heart 105. The electrodes may be electrically coupled to sense amplifiers to sense electrical cardiac signals.

An IMD can be leadless (e.g., a leadless pacemaker, or leadless diagnostic device). The IMDs described are just examples, and it is contemplated that a medical electronic system can be a wearable medical device (e.g., a diagnostic device, loop recorder, or a device to provide therapy). Wearable medical devices can include surface electrodes (e.g., electrodes for skin contact) to sense a cardiac signal such as an electrocardiograph (ECG).

As shown in FIG. 1, a system may include a medical device programmer or other external device 170 that communicates wireless signals 190 with an IMD 110 or wearable medical device, such as by using radio frequency (RF) signals, inductive signals, acoustic signals, conductive telemetry, or other telemetry means. If the medical device is wearable, wired communication can be included.

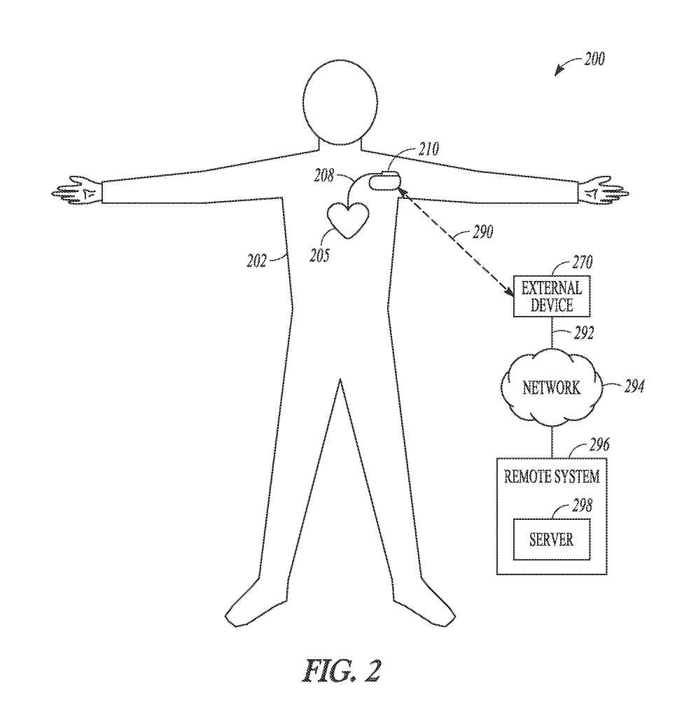

FIG. 2 is an illustration of portions of another system 200 that uses an IMD, wearable medical device, or other ambulatory medical device 210 to provide a therapy to a patient 202. The system 200 typically includes an external device 270 that communicates with a remote system 296 via a network 294. The network 294 can be a communication network such as a phone network or a computer network (e.g., the internet). In some examples, the external device 270 includes a repeater and communicates via the network using a link 292 that can be wired or wireless. In some examples, the remote system 296 provides patient management functions and can include one or more servers 298 to perform the functions. Device communications can allow for remote monitoring for the risk of an acute HF event. Device-based sensor data may provide a continuous indicator of a subject's HF status and can be useful to monitor risk of worsening heat failure.

Medical electronic systems and devices can c de additional physiologic sensors to monitor other physiologic parameters. An example of a physiologic sensor is a thoracic impedance sensor. For instance, to measure thoracic impedance, a specified stimulus signal (e.g., an electrical stimulus of a known current or voltage) can be applied across the thorax region of the patient. A sensed signal (e.g., voltage or current) can be used to determine the impedance, such as by Ohm's Law for example. Thoracic impedance may be intrathoracic impedance if it is measured using electrodes that are implanted somewhere within the thoracic region. For instance, the specified stimulus signal can be applied between cardiac ring electrode 140 and an electrode 111 formed on the housing of the IMD 110. If the IMD is implanted in the pectoral region of the patient, the region between the electrodes spans a significant portion of the thorax region of the subject. Other electrodes useful to measure intrathoracic impedance include other tip or ring electrodes included in implantable cardiac leads (108A, 108B, 108C) or an electrode 155 included in the header of the IMD. An approach to measuring thoracic impedance is described in Hartley et al., U.S. Pat. No. 6,076,015, “Rate Adaptive Cardiac Rhythm Management Device Using Transthoracic Impedance,” filed Feb. 27, 1998, which is incorporated herein by reference in its entirety.

Thoracic impedance may be transthoracic impedance if it is measured using surface electrodes or skin electrodes, such as with a wearable device for example. The electrodes can be positioned so that a substantial portion of the subject's thorax region is between the electrodes. A stimulus signal and a sensing signal are then used to determine the impedance. In certain examples, two surface electrodes are used to apply the stimulus signal and two electrodes are used to sense a signal to determine impedance.

Thoracic impedance information can be used to monitor fluid build-up in the thorax region of the subject. A decrease in thoracic impedance may indicate an increase in interstitial fluid build-up due to pulmonary edema. Most heart failure patients admitted to a hospital have some level of pulmonary congestion. Typically, thoracic impedance information of a subject is collected to establish a reference or baseline impedance value. An assessment of pulmonary congestion for the subject is then determined by the extent of a change from the established baseline. Generally, a relative measurement of thoracic impedance from an established reference is used to provide an assessment for a subject. An absolute measure of thoracic impedance can be an instantaneous measure of thoracic impedance or an impedance measure obtained over a short time frame (e.g., minutes), rather than a relative assessment. The present inventors have determined that trending the value of absolute thoracic impedance can provide useful information for detecting WHF.

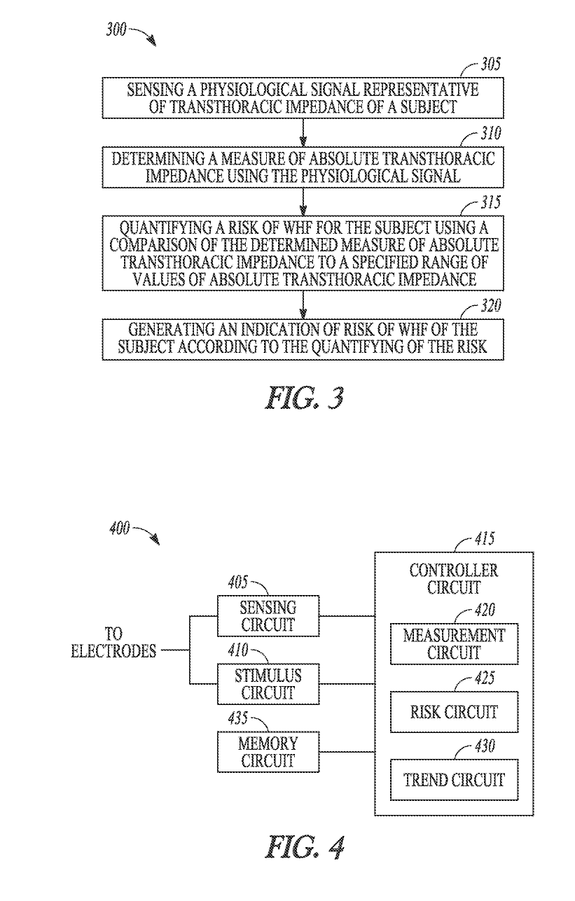

FIG. 3 is a flow diagram of a method 300 of operating an ambulatory medical device to assess the risk that a subject will experience an HF event. At 305, a physiological signal is sensed that is representative of thoracic impedance of a subject. At 310, determining a measure of absolute thoracic impedance is determined using the physiological signal.

At 315, a risk of WHF for the subject is quantified by the medical using a comparison of the determined measure of absolute thoracic impedance to a specified range of values of absolute thoracic impedance. In some examples, the determined measure of absolute thoracic impedance is compared to a specified threshold value of absolute thoracic impedance. In certain variations, the measure of absolute impedance is compared to a specified range of values. Values can be specified by software or by programming a value into the device through a user interface. The specified threshold value identifies the subject as one percent (1%) or less of a specified subject population having the lowest thoracic impedance. This small percentage identifies those subjects at the highest risk of WHF within a specified period of time (e.g., over the next month) and therefore reflects those of the subject population for which physicians should give the most attention.

In an illustrative example, a threshold impedance value of substantially thirty ohms (e.g., 30Ω±5%) or less identifies those subjects at relatively the highest risk of WHF. A smaller highest percentage of the highest risk patients can be determined by using a threshold value of 25Ω or less, or 20Ω or less. A larger percentage of the highest risk patients can be determined by using higher threshold value (e.g., 35Ω or less, 40Ω or less, 50Ω or less, or 60Ω or less).

At 320, an indication of risk of WHF of the subject is generated according to the quantifying of the risk and the indication is provided to a user or process (e.g., a process executing on a computing device). The indication can be used to generate an alert. The alert can be a risk assessment displayed on a programmer, or an alert sent to a server where the alert can be distributed (e.g., over a cellular telephone network or a computer network) to notify caregivers (e.g., a physician). One or both of the indication and the alert can be used to shorten the scheduled time between follow-up visits or examinations for the patient. If the patient is assessed as having lower risk, the device may do nothing. In certain examples, an indication of low risk may be generated which may be displayed on a device or used to lengthen the scheduled time between follow-up visits.

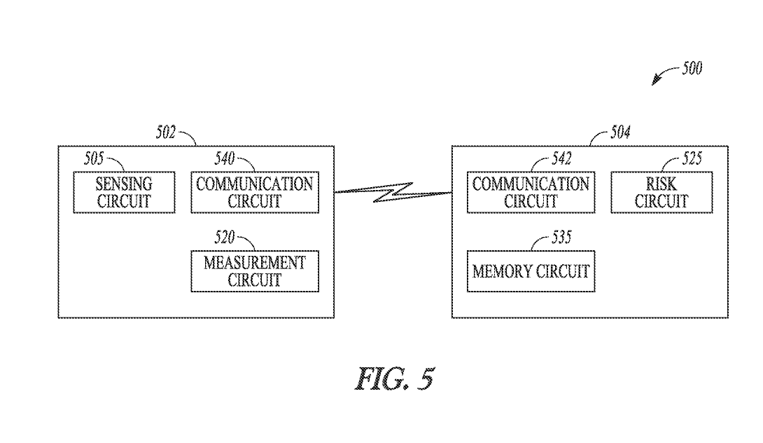

FIG. 4 shows portions of an example of an ambulatory medical device to assess the risk that a subject will experience worsening of their heart failure status within a specified period of time (e.g., within a subsequent week, month, or year). The ambulatory medical device can be implantable or wearable. The device 400 includes a sensing circuit 405 that generates a sensed physiological signal representative of thoracic impedance of a subject. The sensing circuit 405 can include an intrathoracic impedance sensing circuit or a transthoracic impedance sensing circuit. As explained previously, a specified electrical stimulus signal can be applied across the thorax region of the patient, and a voltage or current signal resulting from the stimulus can be used to determine the thoracic impedance. In some examples, the device includes a stimulus circuit 410 to provide the electrical stimulus and the sensing circuit 405 can include one or more sense amplifiers to sense an electrical signal resulting from the electrical stimulus.

The sensing circuit 405 and stimulus circuit 410 can be electrically coupled to electrodes. The device 400 can be wearable and the sensing circuit 405 and stimulus circuit 410 can be electrically coupled to electrodes attachable to the skin surface. A first set (e.g. a pair) of electrodes can be used to provide the stimulus that is sensed by a second set of electrodes. The device 400 can be implantable and the sensing circuit 405 and stimulus circuit 410 can be electrically coupled to electrodes that are implantable, such as the example electrodes of FIG.1. The stimulus circuit 410 can also be used to provide electrical cardiac therapy to the heart of the subject such as electrical pacing therapy or electrical cardioversion/defibrillation therapy. When measuring impedance, the magnitude of the stimulus is less than a magnitude necessary to stimulate tissue.

The device 400 includes a controller circuit 415 electrically coupled to the sensing circuit 405 and the stimulus circuit 410 if any. The controller circuit 415 can include a microprocessor, a digital signal processor, application specific integrated circuit (ASIC), or other type of processor, interpreting or executing instructions in software modules or firmware modules. The controller circuit 415 can include other circuits or sub-circuits to perform the functions described. These circuits may include software, hardware, firmware or any combination thereof. Multiple functions can be performed in one or more of the circuits or sub-circuits as desired.

The controller circuit 415 includes a measurement circuit 420 that determines a measure of absolute thoracic impedance using the sensed physiological signal generated by the sensing circuit 405. The controller circuit 415 also includes a risk circuit 425 that quantifies the risk of WHF for the subject using a comparison of the determined measure of absolute thoracic impedance to a specified threshold value or specified range of values of absolute thoracic impedance. In some examples, the value or values can be stored in memory circuit 435 coupled to or integral to the controller circuit 415, and may identify the subject as belonging to a small fraction of a specific population of subjects having the highest risk of WHF of that subject population. In certain variations, the specified threshold value or specified range of values identifies the subject as one percent or less of the subject population having the highest risk of WHF. The controller circuit 415 generates an indication of risk of WHF of the subject according to the quantifying of the risk.

The risk circuit 425 may use measurement data obtained for a specified period of time in the past (e.g., history data) to quantify the risk of WHF for specified period of time in the near future. For instance, the risk circuit 425 may use one month of history data of absolute thoracic impedance to quantify the risk of WHF over the next month. In another example, the risk circuit 425 may use one month of history data to quantify the risk of WHF over the next year. In still another example, two months of history data may be used to quantify the risk of WHF over the next two years.

The quantifying by the risk circuit 425 can include determining a risk score based on a comparison of the absolute thoracic impedance to the specified range of impedance values. For instance the risk score may increase for smaller values of impedance in the range of impedance values. In certain variations, the quantifying is binary and either an alert may be generated that the subject belongs to the highest risk group when the thoracic impedance satisfies the threshold or no alert is generated.

The sensing circuit 405 can be electrically coupled to different electrodes to determine absolute thoracic impedance using different sensing vectors. Returning to FIG. 1, the sensing vector can include an electrode configured (e.g., by one or more of material, shape and size) for placement in or near the right atrium (RA) of the heart (e.g., any of tip electrode 130, ring electrode 125, defibrillation coil electrode 180, or a ring electrode 185 positioned near the coronary sinus) and the housing or “Can” electrode 111 (RACan). The stimulus vector can include any of the RA electrodes not used in the sensing vector and housing electrode 111 or header electrode 155. In another example, the sensing vector can include an electrode configured for placement in or near the right ventricle (RV) of the heart (e.g., any of tip electrode 135, ring electrode 140, defibrillation coil electrode 175) and the housing electrode 111 (RVCan). The stimulus vector can include any of the RV electrodes not used in the sensing vector and housing electrode 111 or header electrode 155. In still another example, the sensing vector can include an electrode configured for placement in or near the left ventricle (LV) of the heart (e.g., any of electrodes 160 and 165 placed in a coronary vein lying epicardially on LV) and the housing electrode 111 (LVCan). The stimulus vector can include the LV electrode not used in the sensing vector and housing electrode 111 or header electrode 155. Other implantable devices may have different vectors available for use depending on their specific electrode arrangement. For a wearable device, different sense vectors can include different combinations of skin surface electrodes positioned at different locations on the subject.

According to some examples, the sensing circuit 405 can be electrically connectable to a plurality of sensing vectors useable to generate a plurality of physiological signals representative of thoracic impedance. For instance, the device 400 may include a switching circuit (not shown) to electrically couple different combinations of electrodes to the sensing circuit 405. This allows the sensing circuit to sense physiological signals in different directions.

The measurement circuit 420 can determine a plurality of measures of absolute thoracic impedance using the plurality of physiological signals. The risk circuit 425 can then combine the plurality of measures into a single measure of absolute thoracic impedance. In some examples, the risk circuit can combine multiple measures of thoracic impedance linearly. For instance, a combined value of impedance Z can be determined by Z=aX+bY, where X and Y are values of thoracic impedance measured using difference vectors and a,b are constants. In some examples, constants a,b are weights assigned to the vectors. For instance, a measure of thoracic impedance determined using vector LVCan may be weighted higher than a measure generated using a different vector. The combined measure of thoracic impedance Z can be determined as a weighted combination of values X and Y.

As explained previously, the controller circuit 415 generates an indication of risk of WHF of the subject according to the quantified risk. To prevent oversensitivity to measures of absolute thoracic impedance, some filtering may be applied to the measurement. For instance, if the specified threshold value of absolute thoracic impedance is 30Ω or less, small excursions below 30Ω or very short excursions below 30Ω may be filtered out through averaging or by using a time requirement before an alert is generated. Additionally, sharp and sustained increases in absolute thoracic impedance during a specified time window (e.g., a 30 day window) may be an indication that the subject is undergoing diuretic therapy. In this case, an alert generated by the device 400 based on the quantified risk may be modified or reset.

According to some examples, the measure of absolute thoracic impedance is normalized to prevent oversensitivity. In some variations, the controller circuit 415 enables the measurement circuit 420 to perform a measurement of absolute thoracic impedance at a specified time of day (e.g., during the afternoon). In some examples, the absolute thoracic impedance is normalized by comparison to a population of similar subjects. In certain variations, the determined measure of absolute thoracic impedance of a subject is only compared to a subject population of similar size (e,g., one or more of height, weight, chest girth, and the like). In certain variations, the determined measure of absolute thoracic impedance of a subject is only compared to a population of subjects with the same comorbidity such as pulmonary disease. In certain variations, the determined measure of absolute thoracic impedance of a subject is only compared to a population of subjects with similar medical devices (e.g., device model number, medical device lead type, etc.). In certain variations, the determined measure of absolute thoracic impedance of a subject is only compared to a population of subjects with a similar medical device implanted or worn in a similar location. This can be useful to reduce variation due to position of the medical device (e.g., variation in sensing vector length, amount of lung tissue in the sensing vector, etc.).

According to some examples, the measure of absolute thoracic impedance is used to assess risk of WHF when the subject experiences a significant change in thoracic impedance. In certain examples, the measurement circuit 420 determines a baseline measure of thoracic impedance using one or more sensed physiological signals and detecting a change in thoracic impedance from the determined thoracic impedance baseline. The risk circuit 425 quantifies the risk of WHF using the comparison of the determined measure of absolute thoracic impedance when the value of the change in thoracic impedance satisfies a specified change threshold value. Note that this is different from assessing risk using only the change from the baseline. A change from the baseline is used as a trigger to the assessment of absolute thoracic impedance and it is the measure of absolute thoracic impedance that is compared (e.g., to 30Ω) in quantifying the risk. The change from the baseline impedance may be larger (e.g., a change of 100Ω).

The measure of absolute thoracic impedance may be combined with trends of signals from other physiologic sensors to quantify the risk of WHF. An example of a physiologic sensor is a heart sound sensor circuit. Heart sounds are associated with mechanical vibrations from activity of a subject's heart and the flow of blood through the heart. Heart sounds recur with each cardiac cycle and are separated and classified according to the activity associated with the vibration. The first heart sound (S1) is the vibrational sound made by the heart during tensing of the mitral valve. The second heart sound (S2) marks the closing of the aortic valve and the beginning of diastole. The third heart sound (S3) and fourth heart sound (S4) are related to filling pressures of the left ventricle during diastole. A heart sound sensor circuit produces an electrical physiologic signal which is representative of mechanical cardiac activation of the subject. The heart sound sensor circuit can be disposed in a heart, near the heart, or in another location where the acoustic energy of heart sounds can be sensed. In some examples, the heart sound sensor circuit includes an accelerometer disposed in or near a heart. In another example, the heart sound sensor circuit includes an accelerometer disposed in the IMD. In another example, the heart sound sensor circuit includes a microphone disposed in or near a heart.

A heart sound sensor circuit can be electrically coupled to the measurement circuit 420, and the measurement circuit 420 may determine a measure of amplitude of an S3 heart sound using the heart sound signal generated by the heart sound sensor circuit. The risk circuit 425 may quantify the risk of WHF using the determined measure of absolute thoracic impedance and the measured S3 heart sound amplitude. In some examples, the controller circuit 415 includes a trend circuit 430 that trends the amplitude of the S3 heart sound (e.g., over time). The risk circuit 425 to quantifies risk of WHF using the determined measure of absolute thoracic impedance and the generated S3 amplitude trend.

Another example of a physiologic sensor is a respiration sensor circuit. A respiration sensor can produce a respiration signal that includes respiration information about the subject. The respiration signal can include any signal indicative of the respiration of the subject, such as inspiratory volume or flow, expiratory volume or flow, respiratory rate or timing, or any combination, permutation, or component of the respiration of the subject. A respiration sensor circuit can include an implantable sensor such as one or more of an accelerometer, an impedance sensor, a volume or flow sensor, and a pressure sensor.

A respiration sensor circuit can be electrically coupled to the measurement circuit 420 and the measurement circuit 420 may determine respiratory rate of the subject using respiration information. A thoracic impedance signal can be a respiration signal used to identify respiration cycles. The thoracic impedance signal may have a signal component that varies with respiration of the subject. In certain examples, the measurement circuit 420 may determine respiratory rate of the subject using the sensed physiological signal representative of thoracic impedance generated by the sensing circuit 405. When a subject experiences WHF, the subject may have an elevated respiratory rate. The trend circuit 430 may generate a respiratory rate trend (RRT), such as a trend of at least one of a daily respiratory rate maximum value, minimum value, or median value, for example. The risk circuit 425 quantifies the risk of WHF using the deter measure of absolute thoracic impedance and the generated respiratory rate trend. By combining RRT and absolute thoracic impedance (Z), a very small group of patients with extremely high risk of WHF can be identified (e.g., a group defined as RRT≧22 breaths per minute and Z≦30 ohms).

In another example, when a subject experiences WHF, the subject may have a greater variation of respiratory rate within a specified time window. For instance, the subject may have a maximum daily respiratory rate of 26 breaths per minute and a minimum daily respiratory rate of 20 breaths per minute within a month, or a variation of respiratory rate trend (ΔRRT) of 26−20=6 breaths per minute during that month. The risk circuit 425 may quantify the risk of WHF using both the determined measure of absolute thoracic impedance and the determined variation of respiratory rate trend (e.g., a high risk group defined as ΔRRT≧6 breaths per minute and Z≦30 ohms).

Other arrangements of the features shown in FIG. 4 are contemplated. For instance, the sensing circuit 405 and stimulus circuit 410 can be included in the IMD 110 in the example of FIG. 1, with the measurement circuit 420, the risk circuit 425, and the trend circuit 430 included in the external system 170 of FIG. 1. In another illustrative example, the sensing circuit 405, stimulus circuit 410, the measurement circuit 420, can be included in the IMD 210 in the example of FIG. 2, and the risk circuit 425, and the trend circuit 430 included in the remote system 296 of FIG. 2.

FIG. 5 shows portions of an example of a medical device system to assess the risk that a subject will experience worsening of their heart failure status. The system 500 includes a first medical device 502 and a second medical device 504. In some variations, both devices can be ambulatory medical devices. For instance, the first medical device 502 can be implantable and the second medical device 504 can be wearable. In some variations, the first medical device 502 can be an ambulatory medical device (wearable or implantable) and the second medical device 504 can be an external device such as a device programmer or a computer system server. As an illustrative example, the first medical device 502 can be the IMD 110 of the example of FIG. 1 and the second medical device 504 can be the external system 170. In another illustrative example, the first medical device 502 can be the IMD 210 of FIG. 2 and the second medical device 504 can be either the external device 270 or the remote system 296, or the features of the second medical device can be distributed between the external device 270 and the remote system 296 of FIG. 2.

The first medical device 502 includes a sensing circuit 505 that generates the sensed physiological signal representative of thoracic impedance, and a measurement circuit 520 that determines a measure of absolute thoracic impedance using the sensed physiological signal. In certain variations, the measurement circuit 520 can be included in a signal processor of the first medical device 502. As described previously, the sensing circuit 505 may be connectable to multiple sensing vectors and the measurement circuit may combine multiple measures of absolute thoracic impedance into a combined measurement. The first medical device 502 also includes a first communication circuit 540 that communicates information of absolute thoracic impedance to a separate device, such as by wireless telemetry for example.

The second medical device 504 includes a second communication circuit configured to communicate information with the first medical device 502, and a risk circuit 525 to quantify a risk of WHF for the subject. The risk circuit 525 quantifies the risk using a comparison of the determined measure of absolute thoracic impedance to a specified range of values of absolute thoracic impedance. The second medical device 504 may include a trend circuit and may trend other physiological measurements as described previously to assess risk.

The risk circuit 525 generates an indication of risk of WHF of the subject according to the quantified risk. The risk circuit 425 may be electrically coupled to a memory circuit 535 integral to or electrically coupled to the second medical device 504. The memory circuit 535 may include comorbidity information of the subject, or the second medical device 504 may be a server with access to an electronic medical record (EMR) for the subject. The comorbidity information may include information related to renal disease, chronic obstructive pulmonary disease (COPD), diabetes, anemia, etc. The risk circuit 525 may generate a recommendation of therapy to a comorbidity of the subject according to the quantified risk of WHF. In certain examples, the memory circuit stores medication information of the subject. The risk circuit 525 generates a recommended change in titration of medication according to the quantified risk of WHF. For instance, the comorbidity information may indicate that the subject has renal disease. A change in a medication regimen may be recommended to reduce lung fluid (as indicated by absolute thoracic impedance), such as by up-titration of dose diuretics to lower fluid and increase impedance away from the risk detection threshold impedance.

Medication information may be useful to modify any alert generated based on the quantified risk of WHF. An indication of diuretic therapy may be stored in memory 535 or may be included in an EMR. This information may be used to reset or stop the alert or modify information included in the alert.

The several examples described herein show the value of device-based measuring of absolute thoracic impedance to identify those patients that are at the highest for experiencing a heart failure related event.

### ADDITIONAL NOTES AND EXAMPLES

Example 1 can include subject matter (such as an apparatus for coupling to a plurality of electrodes) comprising a sensing circuit configured to generate a sensed physiological signal representative of thoracic impedance of a subject and a controller circuit. The controller circuit can be electrically coupled to the sensing circuit and including: a measurement circuit configured to determine a measure of absolute thoracic impedance using the sensed physiological signal; and a risk circuit configured to quantify a risk of worsening heart failure (WHF) for the subject using a comparison of the determined measure of absolute thoracic impedance to a specified range of values of absolute thoracic impedance, and generate an indication of risk of WHF of the subject according to the quantifying of the risk.

In Example 2, the subject matter of Example 1 optionally includes a memory circuit configured to store a specified range of values of absolute thoracic impedance that identifies the subject as one percent or less of a specified subject population having the highest risk of WHF of the specified subject population.

In Example 3, the subject matter of one or both of Examples 1 and 2 optionally include a risk circuit configured to generate the indication of risk of WHF of the subject when the determined measure of absolute thoracic impedance is substantially equal to thirty ohms (30Ω) or less.

In Example 4, the subject matter of one or any combination of Examples 1-3 optionally includes at least one of: a sensing vector that includes an electrode configured for placement in or near a right atrium of a heart and an electrode incorporated into a housing of the medical device; a sensing vector that includes an electrode configured for placement in or near a right ventricle of a heart and an electrode incorporated into a housing of the medical device; or a sensing vector that includes an electrode configured for placement in or near a left ventricle of a heart and an electrode incorporated into a housing of the medical device. The sensing circuit is optionally configured to sense the physiological signal representative of intra-thoracic impedance using the at least one sensing vector.

In Example 5, the subject matter of one or any combination of Examples 1-4 optionally includes a sensing circuit including a plurality of electrodes to form a plurality of sensing vectors useable by the sensing circuit to generate a plurality of physiological signals representative of thoracic impedance, a measurement circuit configured to determine a plurality of measures of absolute thoracic impedance using the plurality of physiological signals, and a risk circuit configured to combine the plurality of measures into a single measure of absolute thoracic impedance using at least one of a linear combination or a weighted combination.

In Example 6, the subject matter of one or any combination of Examples 1-5 optionally includes a controller circuit configured to enable the measurement circuit to perform a measurement of absolute thoracic impedance at a specified time of day.

In Example 7, the subject matter of one or any combination of Examples 1-6 optionally includes a measurement circuit configured to determine a baseline measure of thoracic impedance using the physiological signal and detecting a change in thoracic impedance from the determined thoracic impedance baseline, and a risk circuit configured to quantify the risk of WHF using the comparison of the determined measure of absolute thoracic impedance when the value of the change in thoracic impedance satisfies a specified change threshold value.

In Example 8, the subject matter of one or any combination of Examples 1-7 optionally includes a heart sound sensor circuit configured to generate a heart sound signal representative of mechanical cardiac activation of the subject, and optionally includes a measurement circuit configured to determine a measure of amplitude of an S3 heart sound using the heart sound signal, and wherein the risk circuit is configured to quantify the risk of WHF using the determined measure of absolute thoracic impedance and the measured S3 heart sound amplitude.

In Example 9, the subject matter of one or any combination of Examples 1-8 optionally includes a trending circuit, a measurement circuit configured to determine respiratory rate of the subject using the sensed physiological signal, a trend circuit configured to generate a trend of at least one of a daily respiratory rate maximum value, minimum value, or median value, and a risk circuit configured to quantify risk of WHF using the determined measure of absolute thoracic impedance and the generated respiratory rate trend.

Example 10 can include subject matter such as a method, a means for performing acts, or a device-readable medium including instructions that, when performed by the device, cause the device to perform acts), or can optionally be combined with the subject matter of one or any combination of Examples 1-9 to include such subject matter, comprising: sensing a physiological signal representative of thoracic impedance of a subject; determining a measure of absolute thoracic impedance using the physiological signal; quantifying, by the medical device, a risk of worsening heart failure (WHF) for the subject using a comparison of the determined measure of absolute thoracic impedance to a specified range of values of absolute thoracic impedance; and generating an indication of risk of WHF of the subject according to the quantifying of the risk and providing the indication to a user or process.

In Example 11, the subject matter of Example 10 optionally includes comparing the determined measure of absolute thoracic impedance to a specified range of values of absolute thoracic impedance that identifies the subject as one percent or less of a specified subject population having the highest risk of WHF of the specified subject population.

In Example 12, the subject matter of one or both of Examples 10 and 11 optionally includes generating the indication when the determined measure of absolute thoracic impedance is substantially equal to thirty ohms (30Ω) or less.

In Example 13, the subject matter of one or any combination of Examples 10-12 optionally includes determining a baseline measure of thoracic impedance using the physiological signal; and detecting a change in thoracic impedance from the determined thoracic impedance baseline, wherein quantifying the risk of WHF for the subject includes quantifying the risk using the comparison of the determined measure of absolute thoracic impedance when the detected change from the determined baseline in thoracic impedance satisfies a specified change threshold value.

In Example 14, the subject matter of one or any combination of Examples 10-13 optionally includes normalizing the determined measure of absolute thoracic impedance for at least one of subject height, subject weight, subject chest girth, medical device location, medical device lead type, or pulmonary disease.

Example 15, can include subject matter (such as a system), or can optionally be combined with the subject matter of one or any combination of Examples 1-9 to include such subject matter, comprising a first medical device and a second medical device. The first medical device optionally includes a sensing circuit configured to generate a sensed physiological signal representative of thoracic impedance of a subject; a measurement circuit electrically coupled to the sensing circuit and configured to determine a measure of absolute thoracic impedance using the sensed physiological signal; and a first communication circuit configured to communicate information of absolute thoracic impedance to a separate device. The second medical device optionally includes a communication circuit configured to communicate information with the first medical device; and a risk circuit configured to quantify a risk of worsening heart failure (WHF) tier the subject using a comparison of the determined measure of absolute thoracic impedance to a specified range of values of absolute thoracic impedance, and generate an indication of risk of WHF of the subject according to the quantifying of the risk.

In Example 16, the subject matter of Example 15 optionally includes the second medical device optionally including a memory circuit configured to store a specified range of values of absolute thoracic impedance that identifies the subject as one percent or less of a specified subject population having the highest risk of WHF of the specified subject population.

In Example 17, the subject matter of one or both of Examples 15 and 16 optionally includes a risk circuit configured to generate the indication of risk of WHF of the subject when the determined measure of absolute thoracic impedance is substantially equal to thirty ohms (30Ω) or less.

In Example 18, the subject matter of one or any combination of Examples 15-17 optionally includes a plurality of electrodes to form a plurality of sensing vectors useable by the sensing circuit to generate a plurality of physiological signals representative of thoracic impedance. The measurement circuit is optionally configured to determine a plurality of measures of absolute thoracic impedance using the plurality of physiological signals, and the risk circuit is optionally configured to combine the plurality of measures into a single measure of absolute thoracic impedance using at least one of a linear combination or a weighted combination.

In Example 19, the subject matter of one or any combination of Examples 15-18 optionally includes a risk circuit configured to generate a recommendation of therapy to a comorbidity of the subject according to the quantified risk of WHF.

In Example 20, the subject matter of Example 19 optionally includes a memory circuit electrically coupled to the risk circuit and configured to store medication information of the subject, wherein the risk circuit is configured to generate a recommended change in titration of medication according to the quantified risk of WHF.

Example 21 can include, or can optionally be combined with any portion or combination of any portions of any one or more of Examples 1-20 to include, subject matter that can include means for performing any one or more of the functions of Examples 1-20, or a machine-readable medium including instructions that, when performed by a machine, cause the machine to perform any one or more of the functions of Examples 1-20.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more,” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An apparatus comprising:
a sensing circuit configured to generate a sensed physiological signal representative of thoracic impedance of a subject;
a controller circuit electrically coupled to the sensing circuit and including:
a measurement circuit configured to determine a measure of absolute thoracic impedance using the sensed physiological signal; and
a risk circuit configured to quantify a risk of worsening heart failure (WHF) for the subject using a comparison of the determined measure of absolute thoracic impedance to a specified range of values of absolute thoracic impedance, and generate an indication of risk of WHF of the subject according to the quantifying of the risk.

2. The apparatus of claim 1, including a memory circuit configured to store a specified range of values of absolute thoracic impedance that identifies the subject as one percent or less of a specified subject population having the highest risk of WHF of the specified subject population.

3. The apparatus of claim 1, wherein the risk circuit is configured to generate the indication of risk of WHF of the subject when the determined measure of absolute thoracic impedance is substantially equal to thirty ohms (30Ω) or less.

4. The apparatus of claim 1, including at least one of:
a sensing vector that includes an electrode configured for placement in or near a right atrium of a heart and an electrode incorporated into a housing of the medical device;
a sensing vector that includes an electrode configured for placement in or near a right ventricle of a heart and an electrode incorporated into a housing of the medical device; or
a sensing vector that includes an electrode configured for placement in or near a left ventricle of a heart and an electrode incorporated into a housing of the medical device, and
wherein the sensing circuit is configured to sense the physiological signal representative of intrathoracic impedance using the at least one sensing vector.

5. The apparatus of claim 1, wherein the sensing circuit includes a plurality of electrodes to form a plurality of sensing vectors useable by the sensing circuit to generate a plurality of physiological signals representative of thoracic impedance, wherein the measurement circuit is configured to determine a plurality of measures of absolute thoracic impedance using the plurality of physiological signals, and wherein the risk circuit is configured to combine the plurality of measures into a single measure of absolute thoracic impedance using at least one of a linear combination or a weighted combination.

6. The apparatus of claim 1, wherein the controller circuit is configured to enable the measurement circuit to perform a measurement of absolute thoracic impedance at a specified time of day.

7. The apparatus of claim 1, wherein the measurement circuit is configured to determine a baseline measure of thoracic impedance using the physiological signal and detecting a change in thoracic impedance from the determined thoracic impedance baseline, and wherein the risk circuit is configured to quantify the risk of WHF using the comparison of the determined measure of absolute thoracic impedance when the value of the change in thoracic impedance satisfies a specified change threshold value.

8. The apparatus of claim 1, including a heart sound sensor circuit configured to generate a heart sound signal representative of mechanical cardiac activation of the subject, wherein the measurement circuit is configured to determine a measure of amplitude of an S3 heart sound using the heart sound signal, and wherein the risk circuit is configured to quantify the risk of WHF using the determined measure of absolute thoracic impedance and the measured S3 heart sound amplitude.

9. The apparatus of claim 1, including a trending circuit,
wherein the measurement circuit is configured to determine respiratory rate of the subject using the sensed physiological signal,
wherein the trend circuit is configured to generate a trend of at least one of a daily respiratory rate maximum value, minimum value, or median value, and
wherein the risk circuit is configured to quantify risk of WHF using the determined measure of absolute thoracic impedance and the generated respiratory rate trend.

10. A method of operating a medical device, the method comprising:
sensing a physiological signal representative of thoracic impedance of a subject;
determining a measure of absolute thoracic impedance using the physiological signal;
quantifying, by the medical device, a risk of worsening heart failure (WHF) for the subject using a comparison of the determined measure of absolute thoracic impedance to a specified range of values of absolute thoracic impedance; and
generating an indication of risk of WHF of the subject according to the quantifying of the risk and providing the indication to a user or process.

11. The method of claim 10, wherein quantifying the risk includes comparing the determined measure of absolute thoracic impedance to a specified range of values of absolute thoracic impedance that identifies the subject as one percent or less of a specified subject population having the highest risk of WHF of the specified subject population.

12. The method of claim 10, wherein generating an indication of risk of WHF of the subject includes generating the indication when the determined measure of absolute thoracic impedance is substantially equal to thirty ohms (30Ω) or less.

13. The method of claim 10, including:
determining a baseline measure of thoracic impedance using the physiological signal; and
detecting a change in thoracic impedance from the determined thoracic impedance baseline, wherein quantifying the risk of WHF for the subject includes quantifying the risk using the comparison of the determined measure of absolute thoracic impedance when the detected change from the determined baseline in thoracic impedance satisfies a specified change threshold value.

14. The method of claim 10, including normalizing the determined measure of absolute thoracic impedance for at least one of subject height, subject weight, subject chest girth, medical device location, medical device lead type, or pulmonary disease.

15. A system comprising:
a first medical device including:
a sensing circuit configured to generate a sensed physiological signal representative of thoracic impedance of a subject;
a measurement circuit electrically coupled to the sensing circuit and configured to determine a measure of absolute thoracic impedance using the sensed physiological signal; and
a first communication circuit configured to communicate information of absolute thoracic impedance to a separate device; and
a second medical device including:
a communication circuit configured to communicate information with the first medical device; and
a risk circuit configured to quantify a risk of worsening heart failure (WHF) for the subject using a comparison of the determined measure of absolute thoracic impedance to a specified range of values of absolute thoracic impedance, and generate an indication of risk of WHF of the subject according to the quantifying of the risk.

16. The system of claim 15, wherein the second medical device includes a memory circuit configured to store a specified range of values of absolute thoracic impedance that identifies the subject as one percent or less of a specified subject population having the highest risk of WHF of the specified subject population.

17. The system of claim 15, wherein the risk circuit is configured to generate the indication of risk of WHF of the subject when the determined measure of absolute thoracic impedance is substantially equal to thirty ohms (30Ω) or less.

18. The system of claim 15, including a plurality of electrodes to form a plurality of sensing vectors useable by the sensing circuit to generate a plurality of physiological signals representative of thoracic impedance, wherein the measurement circuit is configured to determine a plurality of measures of absolute thoracic impedance using the plurality of physiological signals, and wherein the risk circuit is configured to combine the plurality of measures into a single measure of absolute thoracic impedance using at least one of a linear combination or a weighted combination.

19. The system of claim 15, wherein the risk circuit is configured to generate a recommendation of therapy to a comorbidity of the subject according to the quantified risk of WHF.

20. The system of claim 19, including a memory circuit electrically coupled to the risk circuit and configured to store medication information of the subject, wherein the risk circuit is configured to generate a recommended change in titration of medication according to the quantified risk of WHF.
